(19) United States
(12) Patent Application Publication
(10) Pub. No.: US 2023/0158130 A1
(43) Pub. Date: May 25, 2023

(54) Compositions and Methods for Generating Tick Immunity

(71) Applicant: YALE UNIVERSITY, New Haven, CT (US)

(72) Inventors: Erol FIKRIG, Guilford, CT (US); Sukanya NARASIMHAN, New Haven, CT (US)

(21) Appl. No.: 18/056,151

(22) Filed: Nov. 16, 2022

Related U.S. Application Data
(63) Continuation of application No. 16/713,959, filed on Dec. 13, 2019, now abandoned.
(60) Provisional application No. 62/779,912, filed on Dec. 14, 2018.

(51) Int. Cl. A61K 39/00; A61P 33/14

(52) U.S. Cl. CPC A61K 39/0003; A61P 33/14; A61K 2039/55566; A61K 2039/55505

(57) ABSTRACT

In various aspects and embodiments the invention provides methods and compositions for generating tick immunity.

### CROSS-REFERENCE TO RELATED APPLICATIONS

The present application is a continuation of, and claims priority to, U.S. patent application Ser. No. 16/713,959, filed Dec. 13, 2019, which claims priority under 35 U.S.C. § 119(e) to U.S. Provisional Patent Application No. 62/779,912, filed Dec. 14, 2018, which is incorporated herein by reference in its entirety.

### BACKGROUND OF THE INVENTION

Ixodes scapularis ticks transmit bacterial, viral and protozoan pathogens, including Borrelia burgdorferi (the causative agent of Lyme disease), representing some of the major vector-borne infectious diseases in the central and northeastern United States. Lyme disease remains the most common vector-borne illness reported in the United States, and the disease incidence is increasing. Further, the incidence of human infections with tick-borne pathogens such as Anaplasma phagocytophilum, Powassan virus and Babesia microti appears to be on the rise. Recent reports suggest that I. scapularis might also transmit Borrelia miyamotoi in the United States.

Ticks can be co-infected with more than one pathogen, and a tick-bite could potentially result in the simultaneous transmission of multiple pathogens. Tick feeding is pivotal for pathogen transmission. While transmission of B. burgdorferi begins sometime after 24-36 h of tick feeding, other tick-transmitted pathogens including A. phagocytophilum, are transmitted earlier. If feeding could be interrupted within 12-24 h, transmission of multiple pathogens might be thwarted. Currently, there is no vaccine against these tick-transmitted pathogens.

Therefore, there is a need in the art for a strategy to derail tick feeding within the first 24 hours of attachment. This disclosure addresses that need.

### SUMMARY OF THE INVENTION

In one aspect, the invention provides a composition comprising a therapeutically effective amount of at least one tick-salivary protein. In another aspect, the invention provides a method of generating tick immunity in a subject, the method comprising administering to the subject in need thereof, a therapeutically effective amount of the composition of the invention.

In certain embodiments, the therapeutically effective amount of tick-salivary protein is selected from the group consisting of Salp10, Salp14, Salp15, Salp20, Salp 25A, SalpHBP, Salp25D, Salp25B, IsPDIA3, Salp12, SalpC1, SalpC24, P19/Salp19, TSLPI, and TIX. In certain embodiments, the therapeutically effective amount of tick-salivary protein is selected from the group consisting of Salp14, Salp15, Salp 25A, SalpHBP, Salp25D, SalpC1, SalpC24, TSLPI, and TIX. In certain embodiments, the composition comprises at least two salivary proteins. In certain embodiments, the at least two salivary proteins are Salp14 and TSLPI.

In certain embodiments, the composition further comprises an adjuvant to the subject. In certain embodiments, the adjuvant is selected from the group consisting of incomplete Freund's adjuvant, Alum, Addavax (equivalent to MF59), MF59 and AS03.

In certain embodiments, the composition further comprises at least one pharmaceutically acceptable carrier.

In certain embodiments, the composition is formulated for administration by at least one route selected from the group consisting of inhalational, oral, rectal, vaginal, parenteral, intracranial, topical, transdermal, intradermal, intramuscular, subcutaneous, pulmonary, intranasal, buccal, ophthalmic, intrathecal, and intravenous.

In certain embodiments, the subject is a mammal. In certain embodiments, the subject is a human.

### BRIEF DESCRIPTION OF THE DRAWINGS

The following detailed description of selected embodiments of the invention will be better understood when read in conjunction with the appended drawings. For the purpose of illustrating the invention, selected embodiments are shown in the drawings. It should be understood, however, that the invention is not limited to the precise arrangements and instrumentalities of the embodiments shown in the drawings.

FIGS. 1A-1F illustrate that immunity elicited by tick saliva recapitulates tick-resistance phenotype on guinea pigs. (FIG. 1A) Sera from guinea pigs immunized with 20 μl of adult saliva with no adjuvant (Saliva) or Ovalbumin (OVA) or from tick-immune guinea pigs (TIGP) were assessed by ELISA for specific antibodies to tick saliva. About 30 clean I. scapularis nymphs were allowed to engorge on each of 3 Hartley female guinea pigs immunized with 20 μl of adult saliva (Saliva) or Ovalbumin (OVA) or on tick-resistant (Tick-immune) guinea pigs and the following parameters assessed. (FIG. 1B) Visualization of redness at the tick bite sites 24 h post-tick attachment; (FIG. 1C) Erythema over the course of feeding; (FIG. 1D) Rate of tick detachment; (FIG. 1E) Percent recovery of repleted ticks; and (FIG. 1F) Engorgement weights of individual nymphs. Error bars in FIG. 1A and FIGS. 1C-1F represent means±SEM. Significance of differences assessed in: FIG. 1C, and FIG. 1D by 2-way ANOVA with Tukey's multiple comparison test; FIG. 1A, FIG. 1E and FIG. 1F by one-way ANOVA with Tukey's multiple comparison test (*p<0.05;**p<0.005).

FIGS. 2A-2F Illustrate that tick saliva elicits protective immunity in the absence of adjuvant. (FIG. 2A) Sera from guinea pigs immunized with 10 μl of adult saliva with no adjuvant (Saliva) or with adjuvant (Saliva+IFA) or Ovalbumin (OVA) were assessed by ELISA for specific antibodies to tick saliva. About 30 clean I. scapularis nymphs were allowed to engorge on each of 2 Saliva, Saliva+IFA or OVA-immunized female guinea pigs and the following parameters assessed: (FIG. 2B) Visualization of redness at the tick bite sites 24 h post-tick attachment; (FIG. 2C) Erythema over the course of feeding; (FIG. 2D) Rate of tick detachment; (FIG. 2E) Percent recovery of repleted ticks; and (FIG. 2F) Engorgement weights of individual nymphs. Error bars in FIGS. 2A-2D represent means±SEM. Significance of differences assessed in: FIG. 2A by one-way ANOVA with Holm-Sidak test; FIG. 2C, and FIG. 2D by 2-way ANOVA with Tukey's multiple comparison test; FIG. 2E and FIG. 2F by one-way ANOVA with Tukey's multiple comparison test. (*p<0.05; **p<0.005).

FIGS. 3A-3F illustrate that proteins and glycosylations are critical elicitors of tick-resistance. (FIG. 3A) Sera from guinea pigs immunized with 20 μl of adult saliva (Saliva) or Ovalbumin (OVA) or saliva treated with a cocktail of glycosidases (Saliva-deglycosylated) or saliva treated with proteinase K (Saliva-protease) were assessed by ELISA for specific antibodies to tick saliva. About 30 clean I. scapularis nymphs were allowed to engorge on each of 3 Hartley female guinea pigs immunized with Saliva or OVA or Saliva-deglycosylated or Saliva-protease and the following parameters assessed: (FIG. 3B) Visualization of redness at the tick bite sites 24 h post-tick attachment; (FIG. 3C) Erythema over the course of feeding; (FIG. 3D) Rate of tick detachment; (FIG. 3E) Percent recovery of repleted ticks; and (FIG. 3F) Engorgement weights of individual nymphs. Error bars in FIG. 3A, and FIG. 3C through FIG. 3F represent means±SEM. Significance of differences assessed in: FIG. 3C, and FIG. 3D by 2-way ANOVA with Tukey's multiple comparison test; FIG. 3A and FIG. 3E by one-way ANOVA with Tukey's multiple comparison test; FIG. 3F by one-way ANOVA with Dunn's multiple comparison test. (*p<0.05; **p<0.005).

FIGS. 4A-4G illustrate that Salp14 and TSLPI are predominant immunogens in saliva. (FIG. 4A) Sera from guinea pigs immunized with 20 μl of adult saliva (Anti-saliva) or Ovalbumin (Anti-OVA) or saliva with IFA (Anti saliva+IFA) or Ovalbumin with IFA (OVA+IFA) were assessed by ELISA for specific antibodies to tick saliva or to individual recombinant secreted salivary protein antigens listed in Table (FIG. 12). (FIG. 4B) Sera from each of 3 guinea pigs immunized with a cocktail of 20 μg each of recombinant Salp14 and TSLPI (Anti-Salp14/TSLPI) or Ovalbumin (Anti-OVA) were assessed by ELISA for specific antibodies to Salp14 or TSLPI. About 30 clean I. scapularis nymphs were allowed to engorge on each of 3 Hartley female guinea pigs immunized with Salp14/TSLPI or OVA and the following parameters assessed: (FIG. 4C) Visualization of redness at the tick bite sites 24 h post-tick attachment; (FIG. 4D) Erythema over the course of feeding; (FIG. 4E) Rate of tick detachment; (FIG. 4F) Percent recovery of repleted ticks; and (FIG. 4G) Engorgement weights of individual nymphs. Error bars in FIG. 4A, FIG. 4B and FIG. 4D through FIG. 4G represent means±SEM. Significance of differences assessed in: FIG. 4D, and FIG. 4 E by 2-way ANOVA and Sidak's multiple comparison test; FIG. 4F and FIG. 4G by Mann-Whitney non-parametric test. (*p<0.05; **p<0.005).

FIGS. 5A-5F illustrate that immunity elicited by a cocktail of recombinant salivary proteins including Salp14 and TSLPI elicits erythema at tick bite sites. (FIG. 5A) Sera from each of 2 guinea pigs immunized with a cocktail of 20 μg each of recombinant salivary proteins (Anti-Salp cocktail 1) or Ovalbumin (Anti-OVA) were assessed by ELISA for specific antibodies to each of the salivary proteins. About 30 clean I. scapularis nymphs were allowed to engorge on each of 2 Hartley female guinea pigs immunized with Salp cocktail 1 or OVA and the following parameters assessed: (FIG. 5B) Visualization of redness at the tick bite sites 24 h post-tick attachment; (FIG. 5C) percent attached ticks; (FIG. 5D) Erythema over the course of feeding; (FIG. 5E) Rate of tick detachment; (FIG. 5E) Percent recovery of repleted ticks; and (FIG. 5F) Engorgement weights of individual nymphs. Error bars represent means±SEM. Significance of differences assessed in: FIG. 5C, and FIG. 5D by 2-way ANOVA with Sidak's multiple comparison test; FIG. 5E and FIG. 5F by Mann-Whitney test. (*p<0.05; **p<0.005).

FIGS. 6A-6F illustrate that protective immunity elicited by tick saliva is dose dependent. (FIG. 6A) Sera from guinea pigs immunized with 1, 0.1 or 0.01 μl of adult saliva (Saliva) or Ovalbumin (OVA) were assessed by ELISA for specific antibodies to tick saliva. About 30 clean I. scapularis nymphs were allowed to engorge on each of 2 Hartley female guinea pigs immunized with 1, 0.1 or 0.01 μl of adult saliva or Ovalbumin (OVA) and the following parameters assessed: (FIG. 6B) Visualization of redness at the tick bite sites 24 h post-tick attachment; (FIG. 6C) Erythema over the course of feeding; (FIG. 6D) Rate of tick detachment (FIG. 6E) Percent recovery of repleted ticks; and (FIG. 6F) Engorgement weights of individual nymphs. Error bars represent means±SEM. Significance of differences assessed in: FIG. 6A, FIG. 6C, and FIG. 6D by 2-way ANOVA with Tukey's multiple comparison test; FIG. 6E and FIG. 6F by one-way ANOVA with Tukey's multiple comparison test. (*p<0.05; **p<0.005).

FIGS. 7A-7F illustrate that lipids and phosphorylations are not critical elicitors of tick-resistance. (FIG. 7A) Sera from guinea pigs immunized with 15 μl of adult saliva (Saliva) or Ovalbumin (OVA) or saliva treated with lipase (Saliva-lipase) or saliva treated with phosphatase (Saliva-phosphatase) were assessed by ELISA for specific antibodies to tick saliva. About 30 clean I. scapularis nymphs were allowed to engorge on each of 2 Hartley female guinea pigs immunized with Saliva or OVA or Saliva-lipase or Saliva-phosphatase and the following parameters assessed: (FIG. 7B) Visualization of redness at the tick bite sites 24 h post-tick attachment; (FIG. 7C) Erythema over the course of feeding; (FIG. 7D) Rate of tick detachment; (FIG. 7E) Percent recovery of repleted ticks; and (FIG. 7F) Engorgement weights of individual nymphs. Error bars represent means±SEM. Significance of differences assessed in: FIG. 7A, and FIG. 7F by one-way ANOVA with Tukey's multiple comparison test; FIG. 7C, FIG. 7D, and FIG. 7 E by 2-way ANOVA with Tukey's multiple comparison test. (*P<0.05; **P<0.005).

FIGS. 8A-8F illustrate that immunity elicited by recombinant Salp14 and TSLPI generated in the mammalian expression system provides partial tick-resistance, but no significant erythema. (FIG. 8A) Sera from each of 3 guinea pigs immunized with a cocktail of 20 μg each of recombinant Salp14m and TSLPIm (Anti-Salp14m-TSLPIm) or Ovalbumin (Anti-OVA) were assessed by ELISA for specific antibodies to Salp14m or TSLPIm. About 30 clean I. scapularis nymphs were allowed to engorge on each of 3 Hartley female guinea pigs immunized with Salp14m-TSLPIm or OVA and the following parameters assessed: (FIG. 8B) Visualization of redness at the tick bite sites 24 h post-tick attachment; (FIG. 8C) Erythema over the course of feeding; (FIG. 8D) Rate of tick detachment; (FIG. 8E) Percent recovery of repleted ticks; and (FIG. 8F) Engorgement weights of individual nymphs. Error bars represent means±SEM. Significance of differences assessed in: FIG. 8C, and FIG. 8D by 2-way ANOVA with Tukey's multiple comparison test; FIG. 8A, FIG. 8E and FIG. 8F by Mann-Whitney test. (*p<0.05; **p<0.005).

FIGS. 9A-9E illustrate that immunity elicited by a cocktail of recombinant salivary proteins lacking Salp14 and TSLPI does not elicit tick resistance. (FIG. 9A) Sera from each of 2 guinea pigs immunized with a cocktail of 20 μg each of recombinant salivary proteins (Anti-Salp cocktail 2) or Ovalbumin (Anti-OVA) were assessed by ELISA for specific antibodies to each of the salivary proteins. About 30 clean I. scapularis nymphs were allowed to engorge on each of 2 Hartley female guinea pigs immunized with Salp cocktail 2 or OVA and the following parameters assessed: (FIG. 9B) Visualization of redness at the tick bite sites 24 h post-tick attachment; (FIG. 9C) Rate of tick detachment; (FIG. 9D) Percent recovery of repleted ticks; and (FIG. 9E) Engorgement weights of individual nymphs. Error bars represent means±SEM. Significance of differences assessed in: FIG. 9C by 2-way ANOVA with Sidak's multiple comparison 846 test; FIG. 9D and FIG. 9E by Mann-Whitney test. (*p<0.05; **p<0.005).

FIG. 10 illustrates that passive immunization of rabbits with a cocktail of Salp antigens and tick challenge. Engorgement weights of nymphal ticks recovered from TSLPI+P19+TIX-immunized animals decreased compared to that from Ovalbumin-immunized (Ova) animals.

FIG. 11 illustrates that nine secreted Salps used to immunize guinea pigs in IFA (30 μg/antigen/animal). Immunized animals were challenged with 30 clean nymphs. Redness/erythema observed within 24h of tick attachment on 9 Salp-immunized animals (score of ˜2) and no redness on control animals (score of 0).

FIG. 12 is a table showing a list of Ixodes scapularis secreted salivary proteins in Salivary protein (Salp) cocktail 1.

FIG. 13 is a table showing a list of Ixodes scapularis secreted salivary proteins in Salivary protein (Salp) cocktail 2

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, selected materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, “tick-immunity” or “tick-resistance” are used interchangeably and refer to an immune response against antigens involved in tick feeding. This response may include or be characterized by shorter tick feeding times and/or lower engorgement weight. Hosts possessing tick-resistance or tick immunity may be less susceptible to or immune from tick-bite transmitted pathogens and conditions, including but not limited to Lyme disease, Anaplasma phagocytophilum, Powassan virus, A. phagocytophilum and Babesia microti.

As used herein, the terms “tick-salivary protein” or “SALP” may refer to any protein present in tick saliva.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly, the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Methods of Generating Tick Immunity

Without wishing to be limited by theory, the invention is based in part on the discovery that vaccination with tick-salivary proteins can provoke an immune response in mammals against these proteins that interferes with the tick's ability to feed on the host (tick immunity) and therefore to transmit tick borne disease. Therefore, in one aspect, the invention provides a method of generating tick immunity in a subject, the method comprising administering to the subject a therapeutically effective amount of at least one tick-salivary protein. In various embodiments, the therapeutically effective amount of tick-salivary protein is selected from the group consisting of Salp10, Salp14, Salp15, Salp25B, IsPDIA3, Salp12, Salp20, Salp 25A, SalpHBP, Salp25D, SalpC1, SalpC24, P19/Salp19, TSLPI, and TIX. In various other embodiments, the therapeutically effective amount of tick-salivary protein is selected from the group consisting of Salp14, Salp15, Salp 25A, SalpHBP, Salp25D, SalpC1, SalpC24, TSLPI, and TIX. As shown in the figures and discussed further in examples presented herein, these tick-salivary proteins have been demonstrated to effectively generate tick immunity and should be construed as non-limiting examples of tick-salivary proteins useful in the methods of the invention. Immunization with multiple tick-salivary proteins may be useful in provoking a robust immune response. Therefore, in various embodiments, at least two tick-salivary proteins are administered to the subject. In other embodiments, at least three, at least four, at least five, at least six, at least seven, at least eight, or at least nine tick-salivary proteins are administered to the mammal. The effective amount of each of these salivary proteins depends on several factors, including the immunogenicity of the specific protein and is readily determinable by one of ordinary skill in the art in possession of the present disclosure. In various embodiments, a therapeutically effective amount of Salp10, Salp25B, IsPDIA3, Salp12, Salp14, Salp15, Salp20, Salp 25A, SalpHBP, Salp25D, SalpC1, SalpC24, P19/Salp19, TSLPI, and TIX is administered to the subject. In various other embodiments, a therapeutically effective amount of Salp14, Salp15, Salp 25A, SalpHBP, Salp25D, SalpC1, SalpC24, TSLPI, and TIX is administered to the subject. In various embodiments, the method further comprises administering an adjuvant to the subject. Any adjuvant's known in the art may be employed. In certain embodiments, the adjuvant is selected from the group consisting of incomplete Freund's adjuvant, Alum, Addavax (equivalent to MF59, MF59), and AS03. The tick-salivary proteins may be incorporated into a pharmaceutical composition and administered by any convenient route of administration. In various embodiments, the at least one tick-salivary protein is administered by at least one route selected from the group consisting of inhalational, oral, rectal, vaginal, parenteral, intracranial, topical, transdermal, intradermal, subcutaneous, pulmonary, intranasal, buccal, ophthalmic, intrathecal, and intravenous. In various embodiments, the subject is a mammal. In various embodiments the subject is a human.

### Compositions for Generating Tick Immunity

In order to facilitate the generation of tick immunity in a mammal, tick-salivary proteins may be formulated into a composition suitable for administration. In another aspect, the invention provides a composition comprising a therapeutically effective amount of at least one tick-salivary protein.

In various embodiments, the tick-salivary protein selected from the group consisting of Salp10, Salp25B, IsPDIA3, Salp12, Salp14, Salp15, Salp20, Salp 25A, SalpHBP, Salp25D, SalpC1, SalpC24, P19/Salp19, TSLPI, and TIX is administered to the subject. In various other embodiments, the tick-salivary protein is selected from the group consisting of Salp14, Salp15, Salp 25A, SalpHBP, Salp25D, SalpC1, SalpC24, TSLPI, and TIX is administered to the subject. In various embodiments, the composition comprises at least two tick-salivary proteins. In other embodiments, at least three, at least four, at least five, at least six, at least seven, at least eight, or at least nine tick-salivary proteins are included in the composition.

In certain embodiments at least two tick-salivary proteins are selected from the group consisting of Salp14 and TSLPI.

In various embodiments, the composition further comprises an adjuvant. In various embodiments, the composition further comprises and at least one pharmaceutically acceptable carrier. In various embodiments, the composition is formulated for administration by at least one route selected from the group consisting of inhalational, oral, rectal, vaginal, parenteral, intracranial, topical, transdermal, intradermal, subcutaneous, pulmonary, intranasal, buccal, ophthalmic, intrathecal, and intravenous.

### Administration/Dosing

In clinical settings, delivery systems for the compositions described herein can be introduced into a subject by any of a number of methods, each of which is familiar in the art. For instance, a pharmaceutical formulation of the composition can be administered by inhalation or systemically, e.g. by intravenous injection.

The regimen of administration may affect what constitutes an effective amount. The therapeutic formulations may be administered to the subject either prior to or after the manifestation of symptoms associated with the disease or condition. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the composition of the present invention to a subject, preferably a mammal, more preferably a human, may be carried out using known procedures, at dosages and for periods of time effective to treat a disease or condition in the subject. An effective amount of the composition necessary to achieve a therapeutic effect may vary according to factors such as the time of administration; the duration of administration; other drugs, compounds or materials used in combination with the composition; the state of the disease or disorder; age, sex, weight, condition, general health and prior medical history of the subject being treated; and like factors well-known in the medical arts. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the composition without undue experimentation.\ Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds of the invention may be formulated for injection or infusion, for example, intravenous, intramuscular, or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present invention may be in the range of from about 0.001 mg to about 5,000 mg per day, such as from about 0.01 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

Actual dosage levels of the cells in the pharmaceutical formulations of this invention may be varied so as to obtain an amount of the composition that are effective to achieve the desired therapeutic response for a particular subject, composition, and mode of administration, without being toxic to the subject.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out selected embodiments of the present invention, and are not to be construed as limiting in any way the remainder of the disclosure.

### Materials and Methods.

### Ticks and Animals

I. scapularis adults, nymphs and larvae were obtained and maintained in an incubator at 23° C. and 85% relative humidity under a 14-hour light, 10-hour dark photoperiod. 4-5-weeks old female Hartley guinea pigs (Charles River, Mass.) were used to feed nymphal ticks. Female New Zealand white rabbits (Charles River, Mass.) were used to feed female adult ticks essentially as described earlier. Adult tick saliva was collected from engorged adult female ticks using Pilocarpine. Approximately 10 μl saliva/adult tick was obtained and saliva from 40-50 fed adults was pooled, aliquoted and stored at −80° C. prior to use.

### Immunization of Guinea Pigs Against Tick Saliva

4-5-weeks old female Hartley guinea pigs (Charles River, Mass.) were immunized subcutaneously with 20 μl (˜4 μg of protein) of tick saliva in the absence of added adjuvant. The animals were boosted twice at 3-week intervals with 20 μl of tick saliva in the absence of adjuvant. Control animals were immunized with 4 μg of Ovalbumin (Ova) and boosted twice at 3-week intervals in the absence of adjuvant. The animals were bled retro-orbitally 2 weeks after the last boost to obtain 500 μl of blood and the serum separated for use in ELISA experiments to assess saliva-specific antibody titers. At least 2 animals were used in each group and experiments repeated three times. Immunizations with saliva or Ova was also performed in incomplete Freund's adjuvant following the same immunization regimen as described above to determine if the oil-water emulsion-based delivery of saliva would enhance immunity elicited by saliva.

To assess the dose-dependent impact of saliva immunization on tick-resistance, guinea pigs were immunized subcutaneously with 1 μl (˜200 ng), or 0.1 μl (˜20 ng) or 0.01 μl (˜2 ng) of tick saliva without added adjuvant following the same regimen as described above using at least two animals/group.

### Glycosidase, Protease, Phosphorylase or Lipase Treatment of Tick Saliva

To remove glycosylations, 20 μl of tick saliva was incubated with a cocktail of glycosidases that removes both N and O-glycosylations and provided in the EDGLY deglycosidase kit (Sigma, MO). Deglycosylation reaction was conducted under denaturing conditions as recommended by the manufacturer. For protease treatment, saliva was digested with 2.5 Units of proteinase K (Sigma-Alrich) in CutSmart buffer (NEB) and 1% SDS for 1 hour at 37° C. For dephosphorylation saliva was incubated in 1× CutSmart buffer, 5 μl calf intestine alkaline phosphatase and PBS at 37° C. for one hour. For lipase treatment saliva was mixed with 5 μl porcine lipase A (1 mg/ml) and 265 μl of PBS and incubated at 37° C. for one hour. Enzymatically-treated saliva was frozen at −80° C. overnight, and thawed prior to immunization of guinea pigs using immunization regimens described above in the absence of adjuvant. Control animals were similarly immunized with untreated saliva in respective buffers.

### Generation of Recombinant Salivary Proteins (Salps)

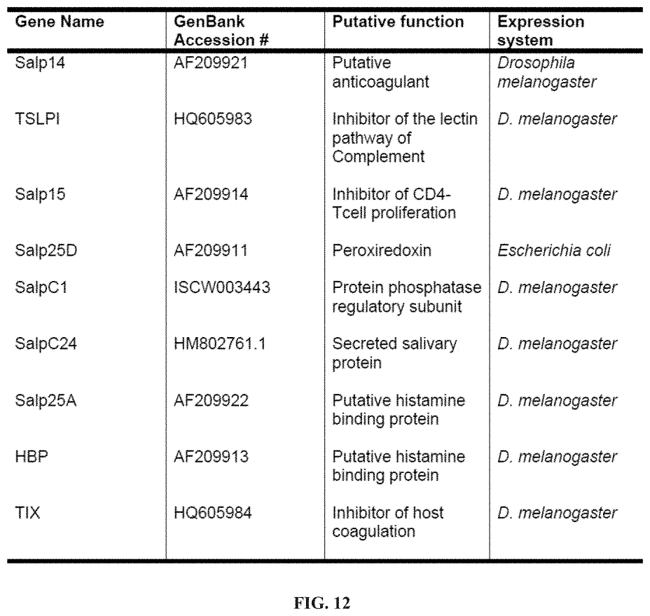

RNA was isolated from salivary glands dissected from I. scapularis ticks fed to repletion and cDNA synthesized according to the manufacture's protocol (iScript cDNA synthesis kit, Bio-RAD). Gene specific primers were used to amplify the mRNA region encoding the mature protein of each Salp listed in Table shown in FIG. 12. Purified amplicons were then cloned into pMT-Bip-V5-HisA vector and recombinant DNA sequenced at the Keck sequencing facility, Yale University, to validate the clones. Recombinant proteins of each Salp was generated using the Drosophila expression system as described earlier and according to the manufacturer's protocol (Invitrogen, CA). To generate recombinant Salp14 and TSLPI in the mammalian expression system (henceforth referred to as Salp14-m and TSLPI-m), the respective amplicons encoding the mature proteins were subcloned into the pEZT-DLUX vector (Addgene, MA) and recombinant DNA sequenced at the Keck sequencing facility, Yale University, to validate the clone. Expression and protein purification of Salp14-m and TSLPI-m were performed using the Expi293 expression system (Thermo Scientific, MA). Protein purity was assessed by SDS-PAGE using 4-20% gradient precast gels (Biorad, CA) and quantified using the BCA protein estimation kit (Thermo Scientific, MA).

### Immunization of Guinea Pigs Against Recombinant Salivary Proteins (Salps)

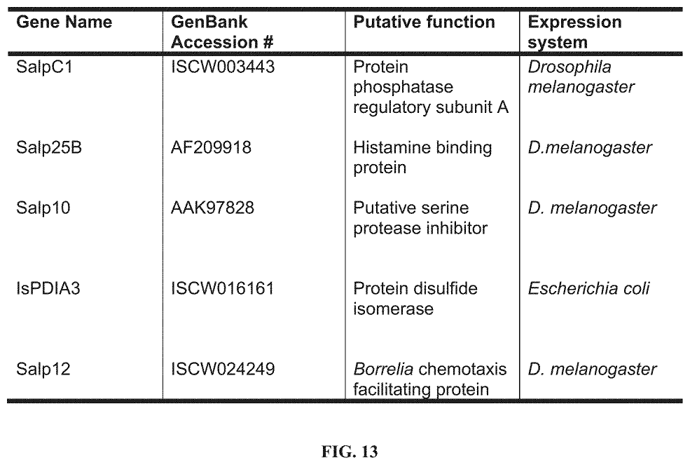

4-5-week-old female Hartley guinea pigs (Charles River, Mass.) were immunized subcutaneously with two individual cocktails of recombinant Salp proteins (listed in Table 1, shown if FIG. 12 Cocktail 1 and Cocktail 2, as shown in FIG. 13) in IFA. The animals were boosted twice at 3-week intervals. Control animals were immunized with Ovalbumin (Ova) and boosted twice at 3-week intervals in the absence of adjuvant. The animals were bled retro-orbitally 2 weeks after the last boost to obtain 500 μl of blood and serum separated for use in ELISA experiments to assess rSalp-specific antibody titers.

### ELISA Assessment of Saliva-Specific or Recombinant Salp-Specific IgG Levels

To assess saliva-specific humoral response 96-well ELISA plates were coated overnight with 500 ng of saliva prepared as described above and incubated with guinea pig anti-saliva sera collected 2-weeks post last immunization and prior to tick challenge at 1:500 or 1:5000 dilution. Bound antibody was detected with HRP-conjugated goat anti-guinea pig IgG and TMB substrate solution (Thermo Scientific, IL). Guinea pig anti-Ova sera collected 2-weeks post last immunization and prior to tick challenge served as control sera. Salp-specific humoral response was similarly assessed using 500 ng of each of the recombinant Salps (FIG. 12) to coat the 96-well ELISA plates and seroreactivity to guinea pig anti-saliva sera or guinea pig anti recombinant Salp cocktail sera.

### Uninfected Tick Challenge of Guinea Pigs

Immunized or naïve guinea pigs were anesthetized by intramuscular injection of ketamine and xylazine mixture and then challenged with 30 nymphal I. scapularis ticks by placing ticks on their shaved backs. Ticks were allowed to attach prior to housing guinea pigs individually in wire-bottom cages with 3 layers of tick containment involving a pan of water below the wire-bottom, a hopper-inclusive lid, and Vaseline grease around the outer edges of the cage. Guinea pigs were monitored daily to monitor the numbers of tick feeding, erythema in skin and to collect any fallen ticks from the water pan and the numbers of ticks obtained was used to calculate percent recovery. Erythema at the tick bite-sites were assessed by two researchers blinded to the experimental groups and scored based on percentage of erythematous tick bite-sites as follows: redness at <10% of tick bite sites: 0.5; redness at 20-50% of tick bite-sites: 1; redness at 50-80% of tick bite sites: 2; redness at =/>80% of tick bite-sites: 3. Repleted ticks were individually weighed using a Sartorius balance to measure engorgement weights as a measure of feeding success.

### Statistical Analysis

In scoring for seroreactivity to saliva or specific salivary antigens, erythema, and rate of tick detachment, the significance of the difference between the mean values of control and experimental groups was analyzed by 2-way ANOVA and Tukey's multiple comparison using with Prism 7.0 software (GraphPad Software, CA). p≤0.05 was considered statistically significant. To assess if percent recovery of ticks and engorgement weights were significantly different between control and experimental groups ordinary ANOVA or two-way ANOVA with Tukey's or Holms-Sidak's multiple comparison or Mann-Whitney test if appropriate was done using Prism 7.0 software. p≤0.05 was considered statistically significant.

### Example 1: Immunization of Guinea Pigs Against Tick Saliva Provokes Robust Tick-Resistance

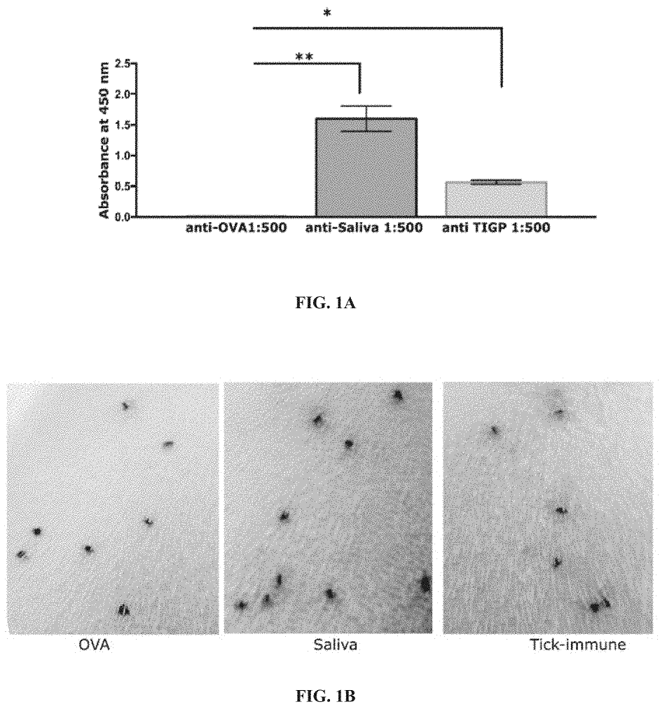

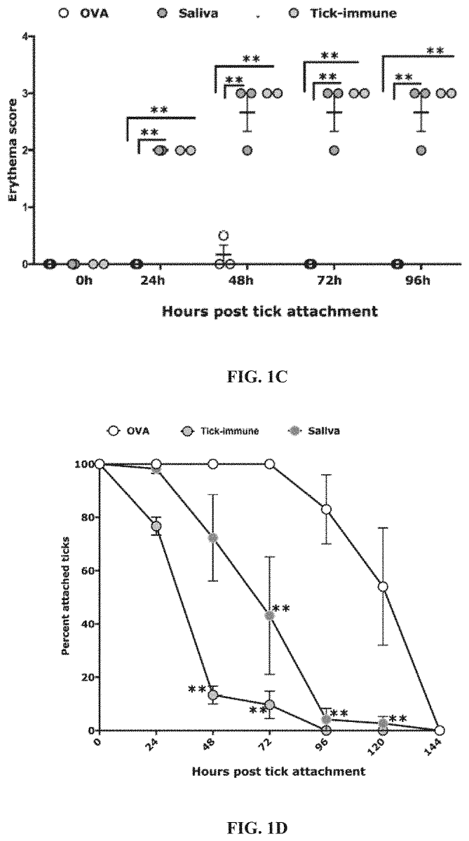

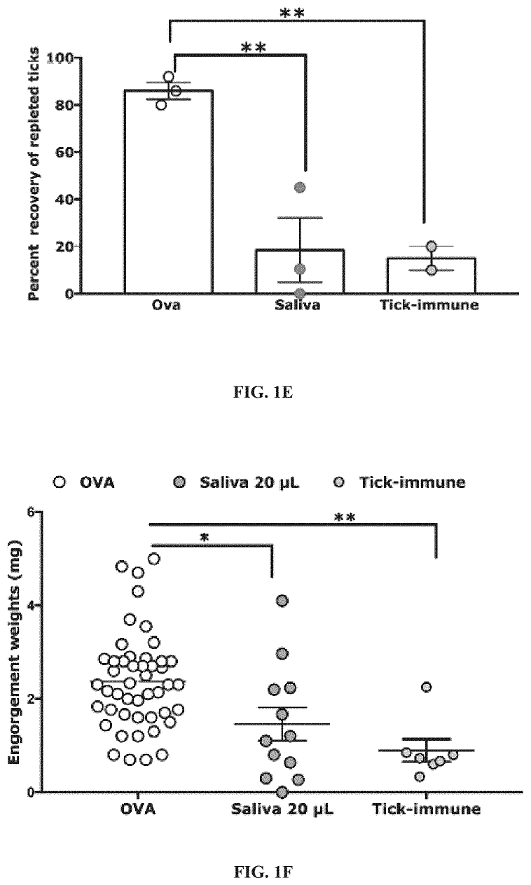

Guinea pigs were immunized with 20 μl (˜4 μg) of adult tick saliva in the absence of any adjuvant and control animals were immunized in parallel with Ovalbumin (Ova), as described elsewhere herein. After the last boost, blood was drawn from each animal and serum levels of antibody specific to tick saliva was confirmed by ELISA prior to challenge of each animal with 30 I. scapularis nymphs (FIG. 1A). Within 24 h of tick attachment, the hallmark redness was observed at the tick bite sites (FIG. 1B) that significantly increased in intensity by 48 h as judged by visible erythema at all tick bite-sites (FIG. 1C) and was comparable to that seen on tick-resistant guinea pigs. Little or no redness was observed in control animals (FIG. 1B-1C). Ticks also detached significantly more rapidly on saliva-immunized animals (FIG. 1D) when compared to that on Ova-immunized animals. Although, tick rejection on tick-resistant guinea pigs was significantly more rapid than that on saliva-immunized animals (FIG. 1D), the recovery of engorged ticks from saliva-immunized animals was comparable to that on tick-resistant animals and was significantly less than that obtained from control animals (FIG. 1E). The engorgement weights of the small number of ticks that fed to repletion on saliva-immunized animals were decreased compared to that on Ova-immunized animals (FIG. 1F).

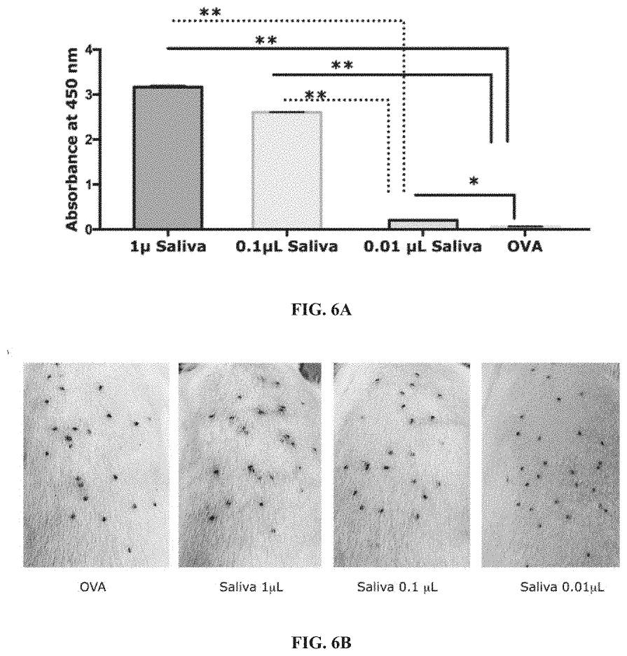

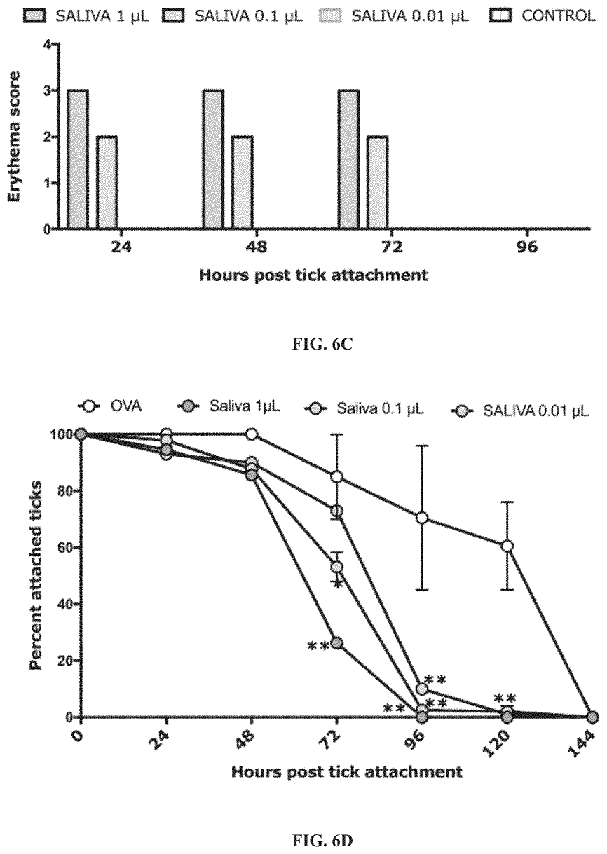

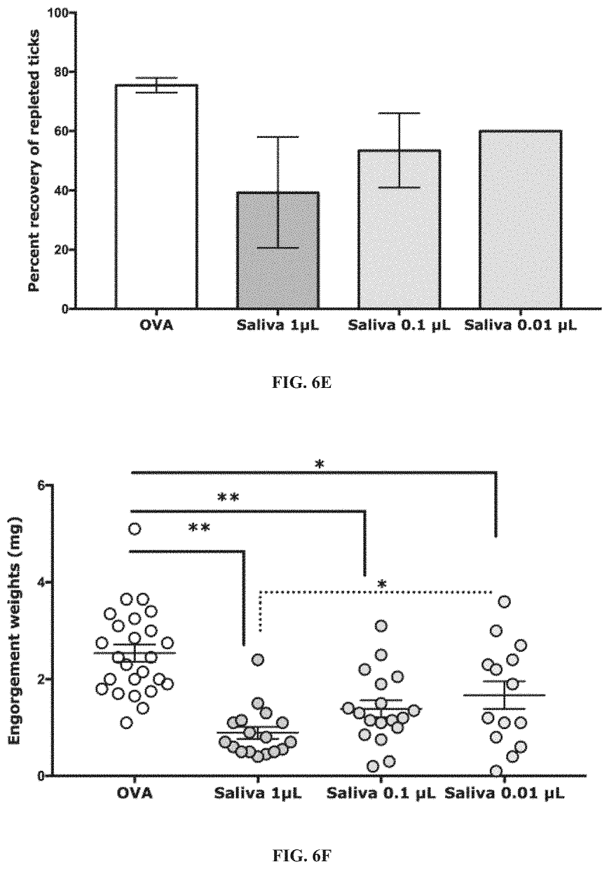

To determine the minimum concentration of saliva that would provide tick resistance, guinea pigs were immunized with decreasing amounts of saliva, as described elsewhere herein. It was observed that immunization of guinea pigs with as low as 1 and 0.1 μL (˜20 ng) of saliva elicited visible redness at tick bite-sites, and 0.01 μL did not provide any redness at the bite-site (FIG. 6B-6C). Although, tick recovery was comparable to that observed in control animals (FIG. 6C), engorgement weights of ticks were significantly reduced on 1, 0.1 and 0.01 μL saliva-immunized compared to ticks that fed control animals. Tick resistance was however significantly reduced when animals were immunized with 0.01 μl (˜2 ng) of tick saliva when compared to that on animals immunized with 1 μL saliva (FIGS. 6A-6F).

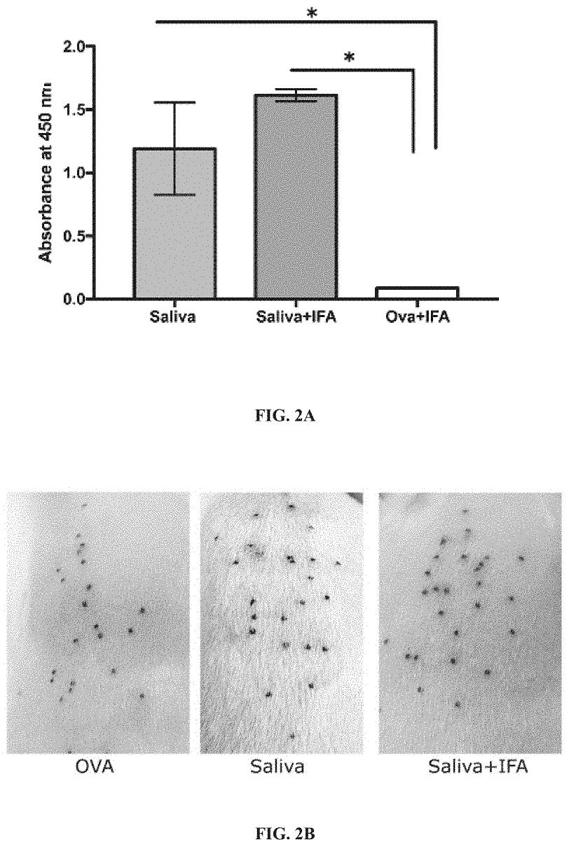

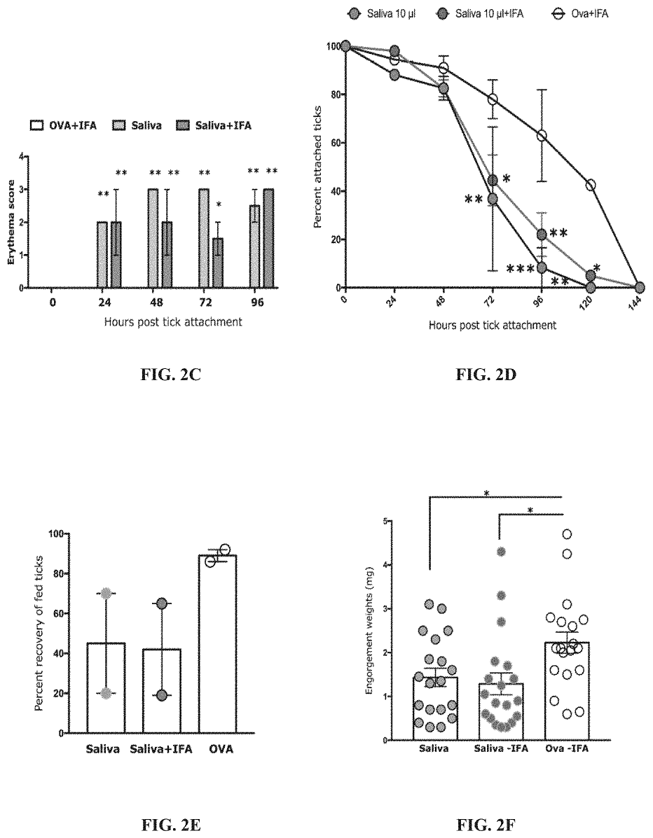

While elicitation of tick-resistance phenotype was achieved without any adjuvant (FIG. 1), it was examined if immunization in presence of Incomplete Freund's (IFA) would enhance the phenotype. Although not a classic adjuvant, by virtue of the oil-water emulsion to form an antigen depot at the injection site and enhance the immune responses, it was reasoned that IFA could boost immune responses to saliva. Animals were immunized with 10 μl (˜2 μg) in presence of IFA and boosted twice as described in, as described elsewhere herein. After the last boost, antibodies specific to tick saliva on the serum was assessed by ELISA (FIG. 2A) and shown to be comparable to that observed in animals immunized with saliva alone (FIG. 2A). Further, upon tick challenge of animals immunized with saliva and IFA the hallmark redness was observed at tick bite-sites, tick rejection and tick recovery that was comparable to that observed in animals immunized with saliva alone (FIGS. 2B-2E). The engorgement weights of ticks that repleted on the immunized animals were also comparably decreased when compared to that on control animals immunized with Ova and IFA (FIG. 2E).

### Example 2: Salivary Proteins and Glycosylations are Critical for Eliciting Tick-Resistance

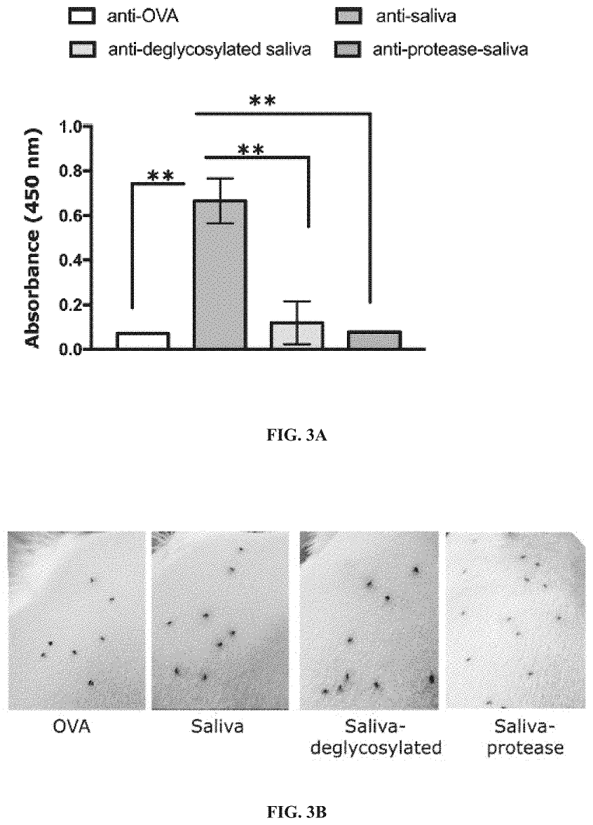

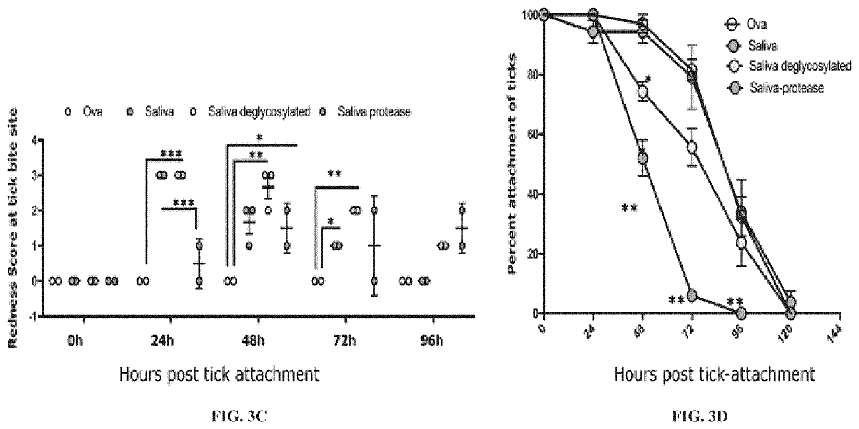

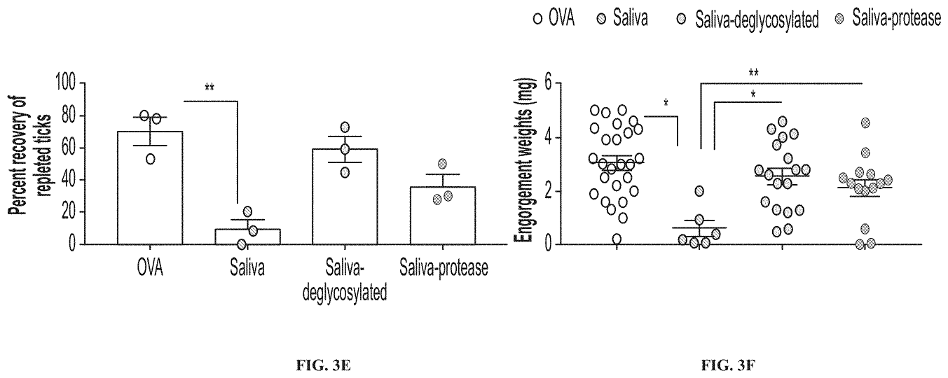

In an effort to determine the components of saliva that play a critical role in eliciting tick-resistance focus was on the salivary proteins, and their post-translational modifications including glycosylations, phosphorylations and lipidations. Saliva 15-20 μl (˜3-4 μg) was treated with protease to enzymatically digest proteins in saliva, with a cocktail of glycosidases to enzymatically deglycosylate salivary proteins, with lipases to remove lipid moieties, or with phosphatase to remove phosphorylations, as described elsewhere herein. Treated or untreated saliva was used to immunize guinea pigs and 10 days after the final boost challenged with ticks as described herein and the development of tick-resistance monitored. ELISA assessment of IgG antibodies specific to saliva showed that glycosidase or protease treatment significantly diminished the reactivity to saliva (FIG. 3A). Protease treatment significantly decreased the development of erythema at the tick bite-sites (FIG. 3B-3C) and abolished the development of tick resistance as seen by tick detachment rate (FIG. 3D), percent recovery of ticks (FIG. 3E) and tick engorgement weights that were comparable to that on control animals (FIG. 3F). Although, tick bite-sites on glycosidase treated saliva-immunized animals showed the hallmark redness that was significantly greater than that on untreated saliva-immunized animals (FIG. 3C), deglycosylation significantly diminished the development of tick resistance as seen by a slower tick detachment rate and higher percent recovery of ticks compared to untreated saliva-immunized animals (FIG. 3D-3E). Engorgement weights of ticks that replete on untreated saliva were significantly decreased compared to ticks that repleted on protease- or glycosidase-treated saliva-immunized animals (FIG. 3F).

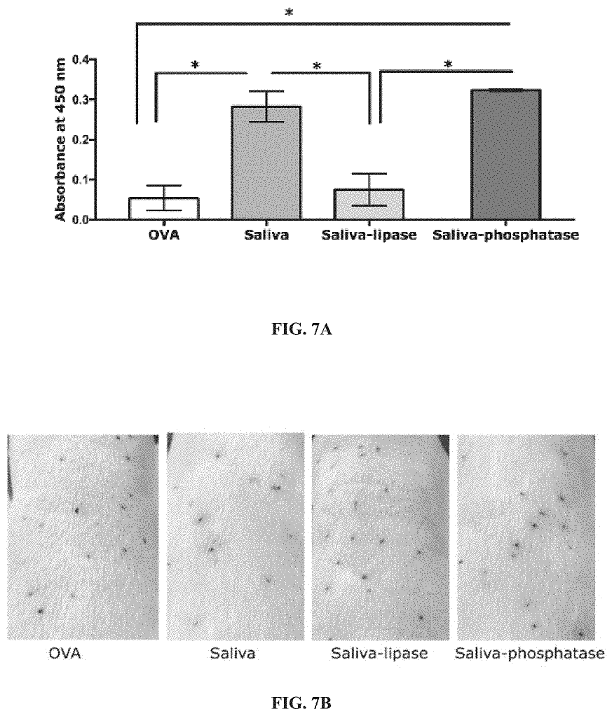

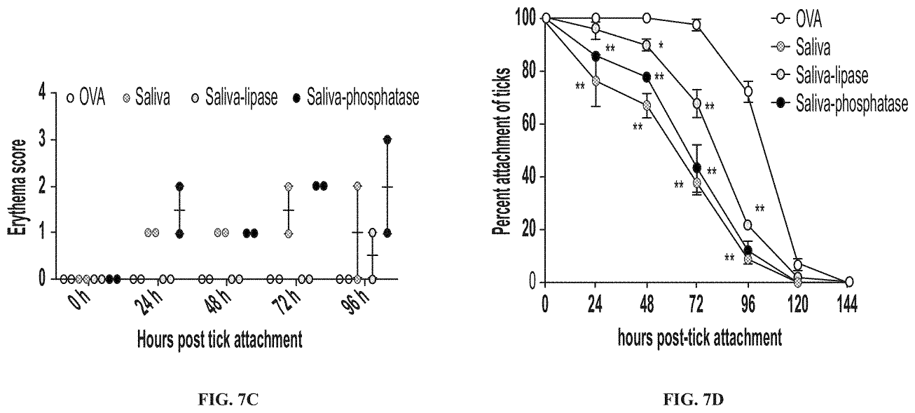

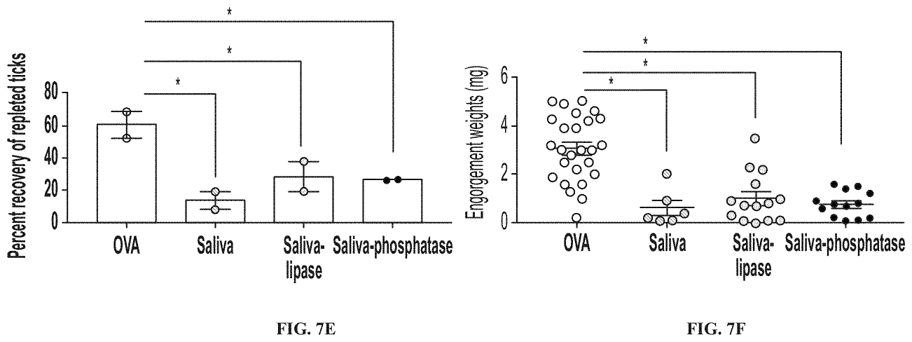

ELISA assessment of IgG antibodies specific to saliva showed that lipase treatment, but not phosphatase treatment, significantly diminished the reactivity to saliva (FIG. 7A). Phosphatase-treated saliva-immunized animals showed all the parameters of tick-resistance including redness at the bite-sites (FIG. 7B-7C), rapid tick detachment, and decreased tick recovery and decreased engorgement weights (FIG. 7D-7F) that was comparable to that on untreated saliva-immunized animals. Lipase treatment prevented the development of erythema at the tick bite-sites (FIG. 7B-7C) but did not significantly impact the elicitation of other parameters of tick-resistance including tick detachment, tick recovery and engorgement weights (FIG. 7D-7F).

### Example 3: Saliva-Immunized Animal Sera Elaborate Robust Humoral Responses to Salp14 and TSLPI

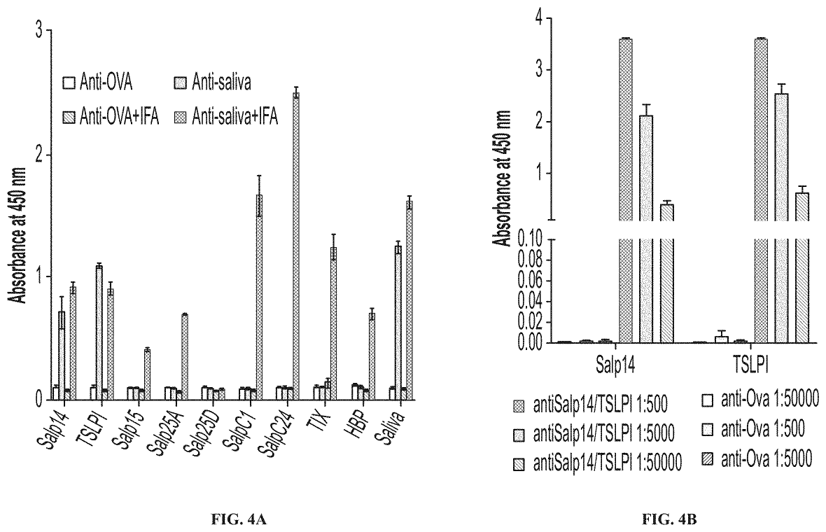

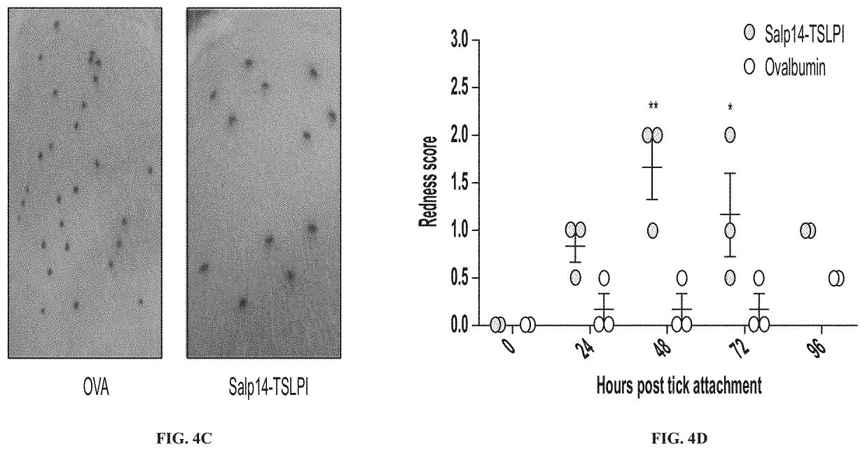

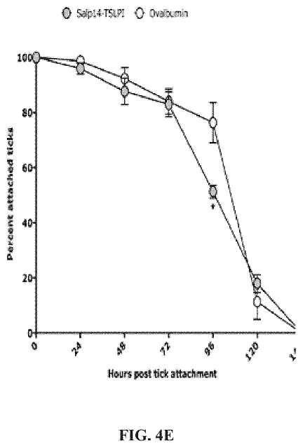

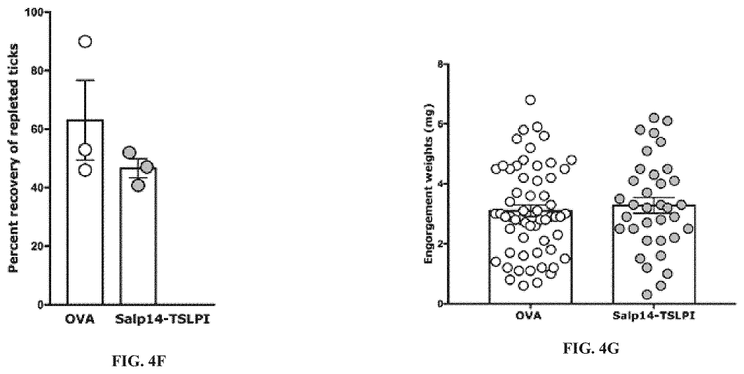

Using various screening approaches it was earlier identified that several salivary proteins (Salps) avidly react with tick-resistant animal sera. Since saliva-immunized animals were significantly protected from tick infestation, seroreactivity of these Salps to saliva-immunized guinea pig sera was assessed by ELISA and western blot using recombinant proteins of these Salps generated in the Drosophila expression system. Recombinant (r) Salp14 and rTSLPI showed strong reactivity to anti-saliva sera when compared to all other recombinant Salps (FIG. 4A). Therefore, guinea pigs were immunized with a cocktail of rSalp14 and TSLPI as described elsewhere herein, and challenged the animals with I. scapularis nymphs to examine if immunity to rSalp14 and rTSLPI was sufficient to elicit tick-resistance. After the last boost, blood was drawn from each animal and presence of antibodies specific to rSalp14 and rTSLPI in the sera was confirmed by ELISA prior to challenge of each animal with ˜30 I. scapularis nymphs (FIG. 4B). The tick bite-sites on rSalp14/TSLPI-immunized animals showed erythema by about 24 h of tick attachment and significantly increased by 48 h when compared to that on control animals (FIG. 4C-4D). Tick attachment was reduced significantly by day 4 (FIG. 4E), and tick recovery was diminished (FIG. 4F). The engorgement weights of the recovered ticks were comparable to that on Ova-immunized animals (FIG. 4G).

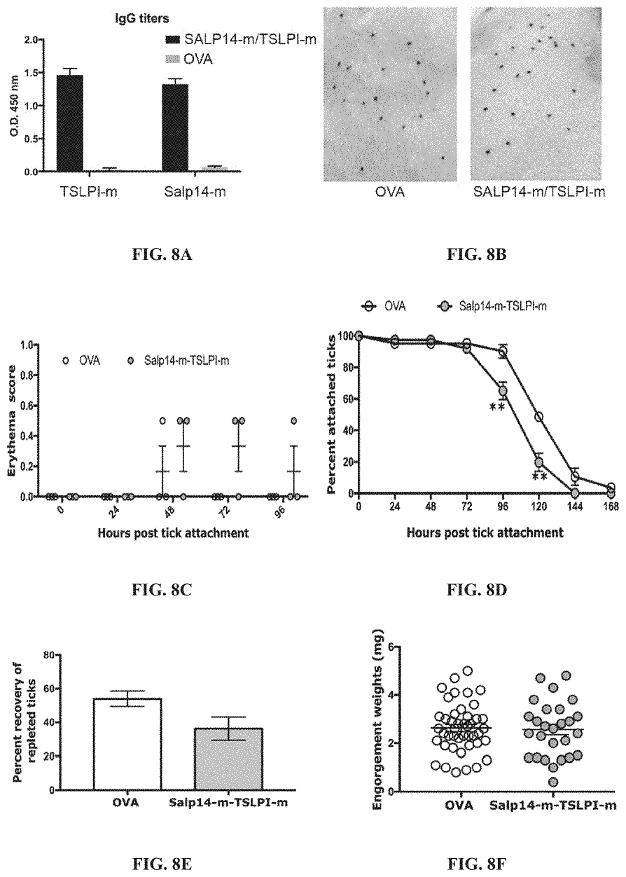

Given that glycosylations on proteins played a significant role in tick rejection, it was also examined if immunization of guinea pigs with Salp14 and TSLPI generated in a mammalian system (rSalp14-m and rTSLPI-m) would impact tick resistance. Guinea pigs were immunized with rSalp14-m/rTSLPI-m and challenged with nymphal ticks, as described elsewhere herein. Presence of antibodies specific to rSalp14m and rTSLPIm in the sera was confirmed by ELISA prior to challenge of each animal with ˜30 I. scapularis nymphs (FIG. 8A). In contrast to the results using rSalp14 and rTSLPI made in a Drosophila expression system, no significant redness was observed at tick attachment sites in the first 3-4 days (FIG. 8B-8C). However, consistent with the previous results, tick attachment was reduced by day 4 (FIG. 8D) when compared to Ova-immunized animals and the recovery of repleted ticks from rSalp14-m/rTSLPI-m was reduced compared to Ova-immunized animals (FIG. 8E). Engorgement weights of the recovered ticks were comparable in both groups (FIG. 8F).

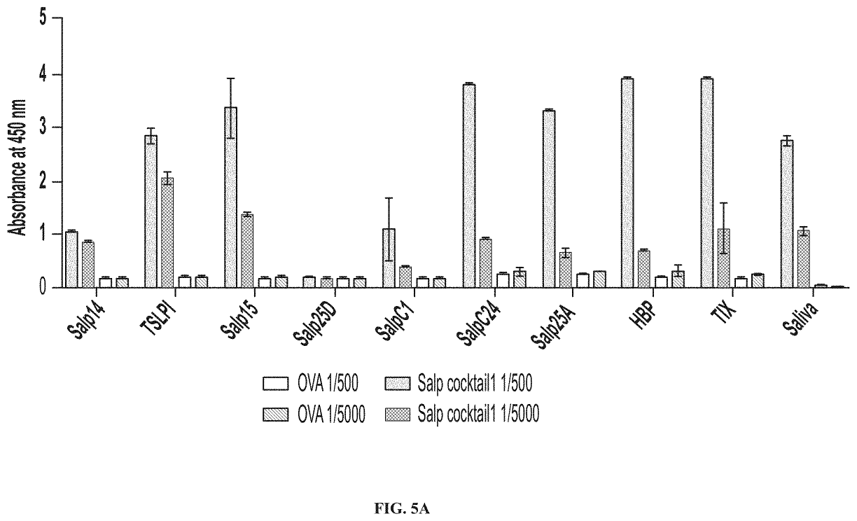

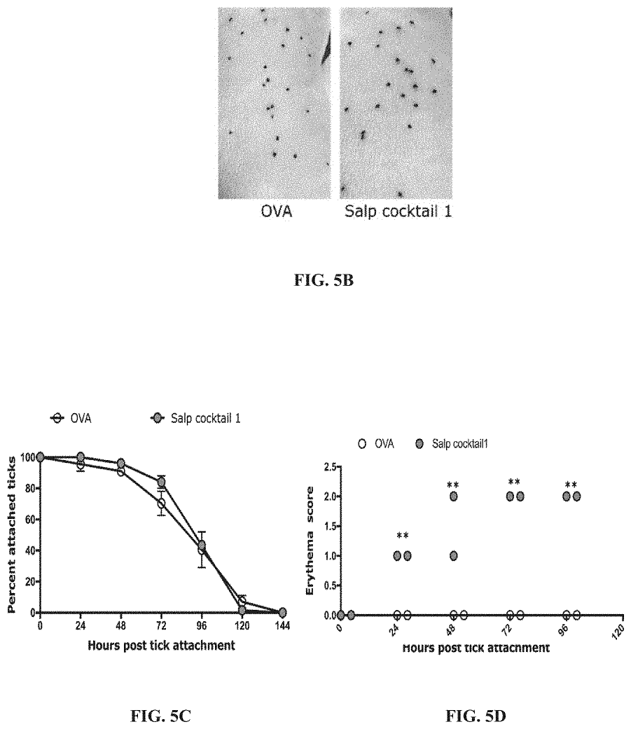

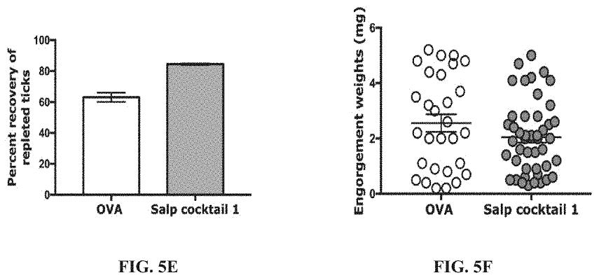

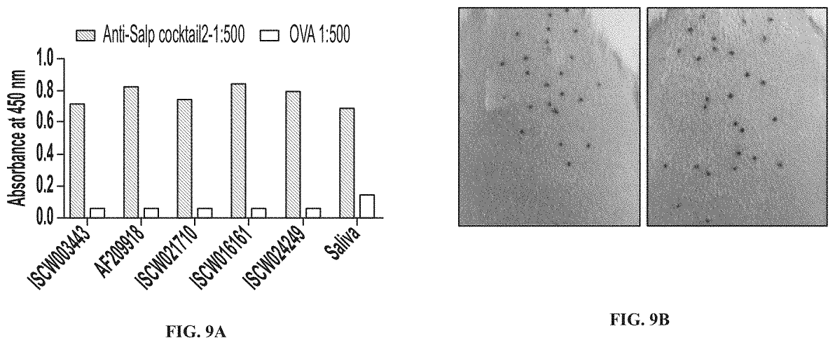

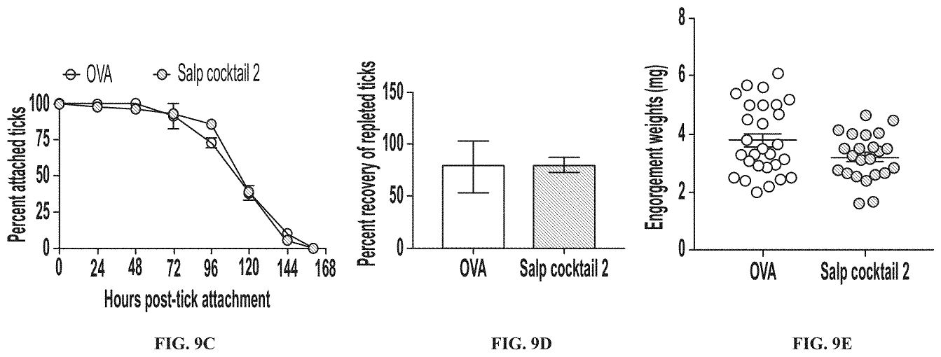

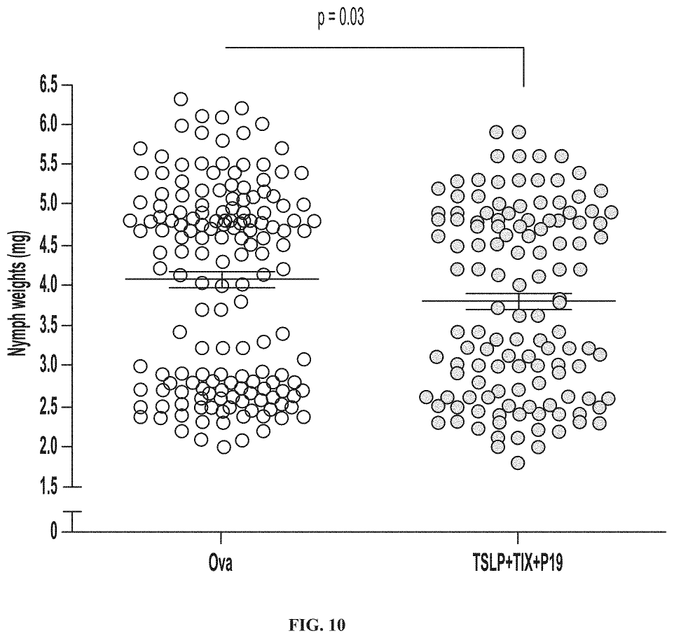

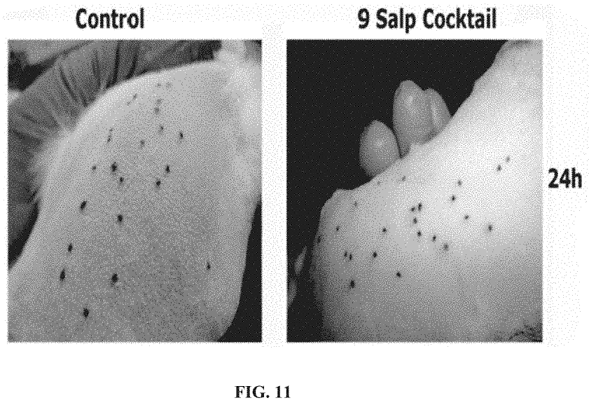

To determine if inflammation at the tick bite-sites on rSalp14/TSLPI immunized animals was unique to Salp14 and TSLPI or if it simply represented reactivity to the respective Salps in tick saliva, animals were also immunized with a cocktail of salivary antigens that did not show reactivity to anti-saliva sera (FIGS. 12-13). Indeed, only cocktails that contained rSalp14 and rTSLPI showed redness at the tick bite-sites (FIG. 5 and FIGS. 9A-9E) and none of the cocktails tested provided tick-resistance phenotype as seen by tick detachment rate, tick recovery and engorgement weights that were comparable to that on Ova-immunized animals (FIG. 5 and FIGS. 9A-9E).

### Example 4

While several proteomic, transcriptomic and functional genomic strategies to develop anti-tick vaccines continue to emerge, The observation that selected non-permissive hosts reject ticks upon multiple infestations remains a robust paradigm to define potential tick vaccine targets to control ticks and prevent tick-transmitted diseases. Over the last several decades, research aimed at understanding the molecular and mechanistic basis of acquired tick-resistance has revealed insights into various host immune components that drive this phenomenon and also invoked several salivary antigens that likely play a role in eliciting tick-resistance. However, the paramount goal of exploiting this phenomenon to develop anti-tick vaccines has not been achieved. Salivary antigens invoked in acquired tick-resistance when tested in vaccine-challenge experiments provided partial protection from tick infestations and pathogen transmission. It was suggested that salivary antigens “exposed” to the host immune responses have likely evolved to counter the immune pressures of the mammalian host and dampened enthusiasm for the search for tick-salivary antigen-based vaccine targets. Given the complexity of the functional genome of ticks, it is likely that multiple factors need to be taken into consideration to fully harness the vaccine potential of tick-salivary antigens. In this study, the guinea pig model were utilized of acquired tick-resistance to examine whether immunity directed against I. scapularis tick saliva elicits robust tick-resistance and to determine salivary components that are critical for eliciting this phenotype.

I. scapularis ticks remain attached to the host for several days and feeding progresses in phases of slow to rapid as feeding culminates in repletion It is now recognized that the tick-salivary proteome is dynamic, shifting in composition during the different phases of feeding to counter the defense responses of the host and successfully feed to repletion. While targeting salivary antigens expressed early in feeding is presumed critical to interrupt tick feeding early, it also suffers from the potential disadvantage of a short window of time for a robust anamnestic response to develop. It was reasoned that salivary proteins secreted into the host throughout the process of feeding are likely to elicit a robust host response. Therefore, tick saliva collected from repleted adults was utilized that is expected to include secreted salivary antigens expressed throughout the course of tick feeding. Tick-salivary and gut proteins have been reported to contain diverse post-translational modifications including glycosylations, phosphorylations and lipidations and these modifications could provide an adjuvant effect. Given that repeated tick infestations deposit natural saliva into the host and elicit a robust immune response that rejects tick feeding, herein it was examined whether immunization of animals with saliva without added adjuvant was sufficient to provoke host immune responses critical for tick rejection. Indeed, when animals immunized with tick saliva were challenged with I. scapularis nymphs, the hallmarks of acquired tick-resistance were observed (FIG. 1) including significant erythema at the tick bite-sites, impaired tick feeding, and diminished tick recovery when compared to control animals. Animals immunized with as low as 20 ng of tick saliva provided partial tick-resistance phenotype as seen by erythema at the bite site, but not tick rejection (FIG. 6A-6F), attesting to the potency of tick saliva. The phenotype was not significantly enhanced when animals were immunized with saliva in presence of adjuvant such as IFA (FIG. 2). Histologic examination of the tick bite-sites on saliva-immunized animals demonstrated increased inflammation characterized predominantly by neutrophils and mononuclear cells and scattered basophils and mast cells.

To determine the role of different components of tick saliva in eliciting tick-resistance the saliva proteins were enzymatically depleted of glycan moieties, phosophorylations or lipid moieties and immunized animals with specific enzyme-treated saliva. The abrogation of the tick resistance phenotype upon depletion of proteins and glycosylations, but not phosphorylations or lipidations suggested that proteins and glycosylations are critical players in eliciting the tick-resistance phenotype (FIG. 3, and FIGS. 8A-8F). These findings, especially the role of glycosylations, emphasize earlier observations that recombinant salivary antigens generated in eukaryotic expression systems were more effective antigens than those made in bacterial expression systems (de la Fuente et al., 2006). There is currently no robust tick cell-line-based protein expression system and most studies utilize insect expression systems such as Drosophila (Anguita et al., 2002) or yeast expression systems such as Pichia pastoris (Kumar et al., 2016). Characterization of tick glycosylation patterns and development of tick-expression systems would help refine tick vaccine antigen and adjuvant development.

Both humoral and cellular immunity is invoked in the elicitation of acquired tick-resistance and transfer of serum from tick-resistant guinea pigs to naïve guinea pigs was shown to confer partial yet significant tick-resistance phenotype. Degranulation of basophils at the tick bite-site, a critical prelude to tick rejection is initiated when specific salivary antigens engage with antigen-specific IgG bound to cognate receptors on basophils, emphasizing the role of humoral immunity in acquired tick-resistance. Earlier studies aimed at defining tick-salivary antigens that react with tick-resistant animal sera had identified several antigens. Of these antigens, it was observed that Salp14, a putative anticoagulant, and TSLPI, an inhibitor of the lectin pathway of the complement system, reacted avidly with anti-saliva sera from saliva-immunized guinea pigs (FIG. 4). The observation that saliva immunization with IFA increased sero-reactivity to several other antigens in addition to Salp14 and TSLPI (FIG. 4A), but did not enhance the tick-resistance phenotype (FIG. 2) suggests that Salp14 and TSLPI are likely among the critical elicitors of tick-resistance.

Salp14 and TSLPI are glycosylated proteins and share 93% identity in the N-terminal region and belong to a family of structurally related proteins. Guinea pigs immunized with a cocktail of recombinant Salp14 and TSLPI (rSalp14/rTSLPI) generated in the Drosophila expression system and challenged with I. scapularis nymphs provided significant erythema at the tick bite-sites, a notable hall mark of tick-resistance, about 24 h post tick attachment (FIG. 4). Despite the significant erythema at the tick bite-sites reminiscent of acquired tick-resistance, immunity against rSalp14-rTSLPI provided modest tick-rejection only around 72-96 hours post tick attachment, and showed a trend towards decreased tick repletion. It is likely that antigens in addition to Salp14 and TSLPI might be required to achieve a more robust tick-resistance phenotype.

Interestingly, when after immunizing guinea pigs with rSalp14-m/rTSLPI-m generated in a mammalian expression system, erythema was not at the bite-site (FIG. 8A-8F), although tick rejection was comparable to that seen on animals immunized with rSalp14/rTSLPI generated in the Drosophila expression system. It is likely that glycosylations on recombinant proteins generated using the mammalian expression system might be less immunogenic in the mammalian host. It is important to note that insect-cell-generated glycosylations by themselves are not contributing to the erythema and that it is a combination of the antigen-glycan epitope. When guinea pigs immunized with cocktails of different subsets of recombinant salivary antigens generated in the Drosophila expression system were challenged with ticks, only cocktails that included rSalp14/rTSLPI provided erythema at the tick bite-site (FIG. 5 and FIGS. 9A-9E).

While immunization against rSalp14/rTSLPI did not provide optimal tick-rejection, it did provide significant erythema at the tick bite-site. Erythema at the tick bite-site is a result of the congregation of immune cells at the bite site that are thought to initiate responses detrimental to tick feeding, including release of histamines from platelets, mast cells and basophils. This would potentially initiate itching of the skin, alert the host to the presence of the tick, and result in removal of the tick. It was reasoned that a vaccine formulation that would alert the host of tick presence would result in rapid tick detection and tick removal that could potentially interrupt tick-transmission of pathogens. It is recognized that that ticks often attach on parts of the body that are not readily visible, but itching and accompanying redness would promote a more rapid surveillance for tick attachment and removal.

It must be bore in mind that tick-resistance phenotype observed upon multiple tick infestations was more effective at rejecting ticks (FIG. 1) compared to that observed on saliva immunized animals. Natural tick infestations might boost the host immune responses additionally by components including the cement cone, and mouth parts directly or indirectly and accelerate tick rejection earlier. Therefore, it is likely that saliva immunizations using higher doses of saliva and using adjuvants might provide more potent tick rejection. Further, saliva obtained from adult ticks is likely not fully reflective of nymphal saliva and could also account, in part, for the differences in the tick-resistance phenotype between saliva-immunized and tick-immune animals.

The demonstration that immunity against tick saliva is sufficient to elicit the hall marks of acquired tick resistance narrows the search to salivary proteins represented in tick saliva and advances in proteomic strategies make this a tractable proposition. All the observations indicate a correlation between humoral responses to specific salivary components, as measured by total IgG, and the elicitation of tick rejection. Erythema, a hall mark of tick resistance, appears to be less critical for tick rejection. It is also evident that the immunogenicity of saliva must be assessed in conjunction with adjuvants to further improve the efficiency of tick-rejection. These observations renew the focus on tick saliva and demonstrate that salivary antigens are key players in eliciting tick resistance, and expand our understanding of the biochemical coordinates on the salivary antigens to enable a viable vaccine design and development.

### Example 5

Earlier observation that non-permissive hosts reject ticks upon multiple tick infestations remains a robust paradigm to define potential tick vaccine targets to control ticks and prevent tick-transmitted diseases. Herein, it is evidenced that immunity elicited by tick saliva in the absence of added adjuvant is sufficient to recapitulate the parameters of tick-resistance including erythema at the tick bite-sites and tick rejection. It is also demonstrated that protein components of tick saliva in conjunction with glycan moieties on these proteins are key elicitors of tick-resistance. These observations redirect our focus on tick-salivary proteins as potential anti-tick vaccine targets and emphasize the need to select appropriate recombinant protein expression systems to achieve optimal vaccine formulations.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of generating tick immunity in a subject, the method comprising administering to the subject in need thereof a therapeutically effective amount of at least one tick-salivary protein.

2. The method of claim 1, wherein the at least one tick-salivary protein is selected from the group consisting of Salp10, Salp25B, IsPDIA3, Salp12, Salp14, Salp15, Salp20, Salp 25A, SalpHBP, Salp25D, SalpC1, SalpC24, P19/Salp19, TSLPI, and TIX.

3. The method of claim 2, wherein the at least one tick-salivary protein is selected from the group consisting of Salp14, Salp15, Salp 25A, SalpHBP, Salp25D, SalpC1, SalpC24, TSLPI, and TIX.

4. The method of claim 1, wherein at least two tick-salivary proteins are administered to the subject.

5. The method of claim 4, wherein the at least two tick-salivary proteins are Salp14 and TSLPI.

6. The method of claim 1, further comprising administering an adjuvant to the subject.

7. The method of claim 6, wherein the adjuvant is selected from the group consisting of incomplete Freund's adjuvant, alum, addavax (equivalent to MF59), MF59, and AS03.

8. The method of claim 1, wherein the at least one tick-salivary protein is administered by at least one route selected from the group consisting of inhalational, oral, rectal, vaginal, parenteral, intracranial, topical, transdermal, intradermal, subcutaneous, pulmonary, intranasal, buccal, ophthalmic, intrathecal, and intravenous.

9. The method of claim 1, wherein the subject is a mammal.

10. The method of claim 9, wherein the subject is a human.

11. A composition comprising a therapeutically effective amount of at least one tick-salivary protein.

12. The composition of claim 11, wherein the at least one tick-salivary protein is selected from the group consisting of Salp10, Salp25B, IsPDIA3, Salp12, Salp14, Salp15, Salp20, Salp 25A, SalpHBP, Salp25D, SalpC1, SalpC24, P19/Salp19, TSLPI, and TIX.

13. The composition of claim 12, wherein the at least one tick-salivary protein is selected from the group consisting of Salp14, Salp15, Salp 25A, SalpHBP, Salp25D, SalpC1, SalpC24, TSLPI, and TIX.

14. The composition of claim 11, wherein the composition comprises at least two tick-salivary proteins.

15. The composition of claim 14, wherein the at least two salivary proteins are Salp14 and TSLPI.

16. The composition of claim 11, further comprising an adjuvant.

17. The composition of claim 16, wherein the adjuvant is selected from the group consisting of incomplete Freund's adjuvant, alum, addavax (equivalent to MF59), MF59, and AS03.

18. The composition of claim 11, wherein the composition further comprises at least one pharmaceutically acceptable carrier.

19. The composition of claim 11, wherein the composition is formulated for administration by at least one route selected from the group consisting of inhalational, oral, rectal, vaginal, parenteral, intracranial, topical, transdermal, pulmonary, intranasal, buccal, ophthalmic, intrathecal, and intravenous.
